(19) United States
(12) Patent Application Publication
(10) Pub. No.: US 2022/0152417 A1
(43) Pub. Date: May 19, 2022

(54) MASK DEVICE FOR SKINCARE USING LIGHT

(71) Applicant: IDEABRIDGE STARTUP VENTURE SPECIALIZED PRIVATE EQUITY FUND, Seoul (KR)

(72) Inventor: Ji Na KIM, Sejong (KR)

(21) Appl. No.: 17/436,362

(22) PCT Filed: Mar. 27, 2020
(86) PCT No.: PCT/KR2020/004189

§ 371 Date: Sep. 3, 2021

(30) Foreign Application Priority Data
Mar. 27, 2019 (KR) 10-2019-0035240

(51) Int. Cl. A61N 5/06

(52) U.S. Cl. CPC A61N 5/0616; A61N 2005/0647; A61N 2005/0663; A61N 2005/0665; A61N 2005/0659; A61N 2005/0652

(57) ABSTRACT

The present invention relates to a mask device for skincare using light. The mask device for skincare using light includes: an outer mask unit; an inner mask unit located to be spaced apart from the outer mask unit; and a plurality of optical sources located between the outer mask unit and the inner mask unit and configured to output light towards the inner mask unit. The inner mask unit includes a substrate including a first surface having a plurality of recesses and a second surface located opposite to the first surface, and the optical sources are located to face the recesses, respectively.

### TECHNICAL FIELD

The present disclosure relates to a mask device for skincare using light, and more particularly, to a mask device for skincare using light used for a face.

### BACKGROUND ART

A mask device using light for skincare such as skin regeneration and wrinkle relief by irradiating a user's skin with light having a predetermined wavelength has been developed and produced.

However, a mask device using light of a related art has a problem in that light emitted from a light source is not uniformly irradiated to the user's face.

### RELATED ART DOCUMENT

### Patent Document

Korean Patent Laid-open Publication No. 10-2019-0070477 (Publication date: Jun. 21, 2019, entitled: Facial skin management system)

### DISCLOSURE

### Technical Problem

An aspect of the present disclosure is to improve uniformity of light irradiated toward a user's face.

Another aspect of the present disclosure is to improve performance of a mask device for skincare using light.

### Technical Solution

According to an aspect of the present disclosure, there is provided a mask device for skincare using light includes: an external mask unit; an internal mask unit located to be spaced apart from the external mask unit; a plurality of light sources located between the external mask unit and the internal mask unit and configured to output light toward the internal mask unit, wherein the internal mask unit includes a substrate including a first surface having a plurality of concave portions and a second surface located opposite to the first surface, and the plurality of light sources are located to face the plurality of concave portions, respectively.

The plurality of concave portions may have a light diffusing function.

The substrate may be formed of a material that transmits light.

The internal mask unit may further include a photocatalyst layer located on at least one of the first surface and the second surface of the substrate.

The photocatalyst layer may contain at least one transition metal fine particle.

The at least one transition metal fine particle may contain at least one of a transition metal and a transition metal compound.

The internal mask unit may further include a reflective layer located on the second surface of the substrate.

The internal mask unit may further include a UV blocking layer located between the second surface of the substrate and the reflective layer.

The mask device may further include a UV blocking layer located between the second surface of the substrate and the reflective layer.

The second surface of the substrate may be a flat surface.

Each of the plurality of light sources may include at least one light emitting device.

The light emitting device may be one of a red light emitting device, a green light emitting device, a yellow light emitting device, and an infrared light emitting device.

Operating frequencies of the red light emitting device, the green light emitting device, the yellow light emitting device, and the infrared light emitting device may all be the same.

The operating frequency may be 130 Hz to 170 Hz.

At least one of the red light emitting device, the green light emitting device, the yellow light emitting device, and the infrared light emitting device may have an operating frequency different from those of the other light emitting devices.

The operating frequency of the red light emitting device may be 50 Hz to 90 Hz, and the operating frequencies of the green light emitting device, the yellow light emitting device, and the infrared light emitting device may be 130 Hz to 170 Hz.

The mask device may further include a UV blocking layer located on the second surface of the substrate.

### Advantageous Effects

According to the features of the present disclosure, the light emitted from the light source unit is diffused by a light diffusion function based on the concave portion so that the light from the light source unit is emitted to the entire face, so that the entire face may be irradiated with light.

Also, by a reflective plate attached to the substrate, light reflected by the skin and output toward the light source is reflected back toward the skin. Accordingly, the amount of light irradiated to the skin is increased.

Furthermore, a skin improvement effect is further improved by the photocatalyst layer, and ultraviolet rays irradiated to the skin are blocked by the UV blocking layer.

### DESCRIPTION OF DRAWINGS

FIG. 1 is a side view of a mask device for skincare using light according to an embodiment of the present disclosure.

FIG. 2 is a front perspective view of a mask device for skincare using light according to an embodiment of the present disclosure.

FIG. 3A is a front exploded perspective view of a mask device for skincare using light according to an embodiment of the present disclosure.

FIG. 3B is a rear exploded perspective view of a mask device for skincare using light according to an embodiment of the present disclosure.

FIG. 4 is a partial rear perspective view showing a state in which a wearing unit is mounted on an internal mask unit of a mask device for skincare using light according to an embodiment of the present disclosure.

FIG. 5 is a cross-sectional view of a portion of an internal mask unit of a mask device for skincare using light according to an embodiment of the present disclosure.

FIG. 6 is a view showing a connection state of a face mask and an operating unit of a mask device for skincare using light according to an embodiment of the present disclosure.

FIG. 7 is a view conceptually illustrating a state in which light irradiated from a light source unit of a face mask device according to an embodiment of the present disclosure is irradiated to a user's skin through a diffusion lens.

FIG. 8 is a view illustrating a concept in which light reflected by a user's skin is reflected by the reflective layer and re-entered toward the user's skin in the face mask device according to an embodiment of the present disclosure.

FIG. 9 is a plan view of a light source unit used in a face mask device according to an embodiment of the present disclosure.

FIG. 10 is a partial cross-sectional view of another example of an internal mask unit of a mask device for skincare using light according to an embodiment of the present disclosure.

FIGS. 11A and 11B are a plan view of a substrate to which a plurality of light sources are attached and a view illustrating an image of the substrate imaged when the light sources are turned on, respectively, according to Comparative Examples.

FIGS. 12A and 12B are a plan view of a substrate to which a plurality of light sources are attached and a view illustrating an image of the substrate photographed when the light sources are turned on, respectively, according to Examples of the present disclosure.

### BEST MODES

Hereinafter, embodiments of the present disclosure will be described in detail with reference to the accompanying drawings. In describing the present disclosure, if it is determined that a detailed description of known functions and components associated with the present disclosure unnecessarily obscure the gist of the present disclosure, the detailed description thereof will be omitted. The terms used henceforth are used to appropriately express the embodiments of the present disclosure and may be altered according to a person of a related field or conventional practice. Therefore, the terms should be defined on the basis of the entire content of this specification.

Technical terms used in the present specification are used only in order to describe specific exemplary embodiments rather than limiting the present disclosure. The terms of a singular form may include plural forms unless referred to the contrary. It will be further understood that the terms “comprise” and/or “comprising,” when used herein, specify the presence of stated features, integers, steps, operations, elements, and/or components, but do not preclude the presence or addition of one or more other features, regions, integers, steps, operations, elements, components, and/or groups thereof.

Hereinafter, a mask device for skincare using light according to an embodiment of the present disclosure will be described with reference to the accompanying drawings.

The mask device for skincare using light in this embodiment may be, for example, a mask device for skincare using light worn on a user's face, but is not limited thereto and may be applied to a mask device for skincare using light worn on other body parts, such as the neck.

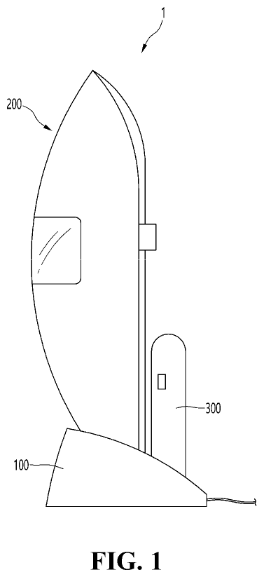

Referring to FIG. 1, the mask device 1 for skincare using light of this example includes a holder 100, a mask (hereinafter, the mask is referred to as a ‘face mask’ worn on the face) 200, and an operating unit 300 for operating the face mask 200.

The holder 100 serves to allow the face mask 200 to be located therein, and a lower portion of the face mask 200 is inserted into a holding recess (not shown) formed in the holder 100 and located.

Therefore, when not used, the face mask 200 is stably located on the holder 100, so space consumption due to the face mask 200 not in use may be minimized and a possibility that the face mask 20 is damaged is prevented.

The face mask 200 may be formed to cover the entire face of the user, and may have a curved structure based on a surface contour of the face.

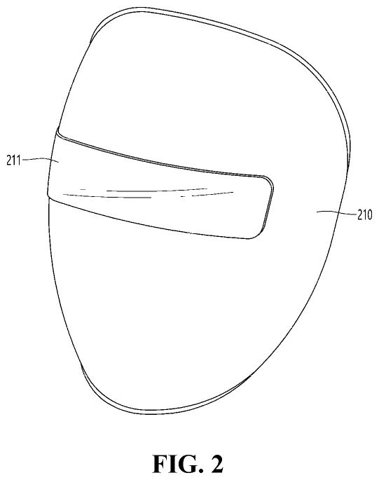

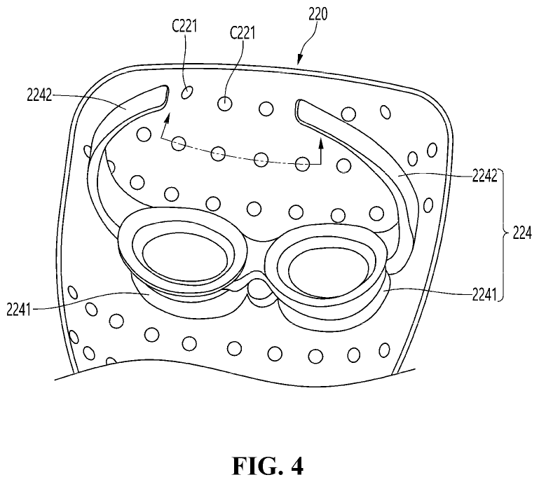

As shown in FIGS. 2 to 5, the face mask 200 may include an external mask unit 210, an internal mask unit 220, and a light source unit 230 located between the external mask unit 210 and the internal mask unit 220 and including a plurality of light sources 231.

The external mask unit 210 is a portion exposed to the outside and may be formed of a light and solid material such as aluminum or plastic.

Both an outer surface (i.e., a surface exposed to the outside) of the external mask unit 210 and an inner surface, which is a surface opposite to the outer surface and adjacent to the internal mask unit 220, may be formed as flat surfaces without concave and convex portions.

The external mask unit 210 has a pair of openings completely penetrating through the external mask unit 210 and exposing the user's eyes. These openings are blocked by a transparent protective plate 211 through which light is transmitted.

Therefore, the user may check an external situation, etc. through the protective plate 211 in a state of wearing the face mask 200 and may also read books or magazines, so a user who uses the mask device for skincare using light of this example may have good time without being bored during the use of the face mask 200.

In addition, since the protective plate 211 prevents an inflow of external dust into the eyes, user convenience is further improved.

A pair of protrusions 212 protruding toward the internal mask unit 220 may be located near each opening of the external mask unit 210.

The internal mask unit 220 may be located to face the external mask unit 210 on the opposite side of the external mask unit 210, and face the user's face adjacently when the face mask 200 is worn.

This internal mask unit 220 is spaced apart from the external mask unit 210 and is coupled to the external mask unit 210. Accordingly, an empty space corresponding to a distance spaced apart from each other may be located between the external mask unit 210 and the internal mask unit 220, and the light source unit 230 may be located in the empty space.

This internal mask unit 220 may include a substrate 221, a reflective layer 222 located on one surface of the substrate 221 (e.g., an outer surface adjacent to the user's face), a photocatalyst layer 223 located on the other surface (e.g., inner surface located opposite to one surface and adjacent to the light source unit 230) of the substrate 221, and a wearing unit 224 for wearing the face mask 200.

In addition, the internal mask unit 220 may include a pair of openings OP220 located to correspond to the pair of openings of the external mask unit 210 and completely penetrating the internal mask unit 220.

Therefore, when the external mask unit 210 and the internal mask unit 220 are coupled, each protrusion 212 of the external mask unit 210 may protrude through each opening 220 of the internal mask unit 220 corresponding thereto.

As such, the wearing unit 224 may be mounted on the protrusion 212 of the external mask unit 210 protruding through an opening OP220 of the internal mask unit 220.

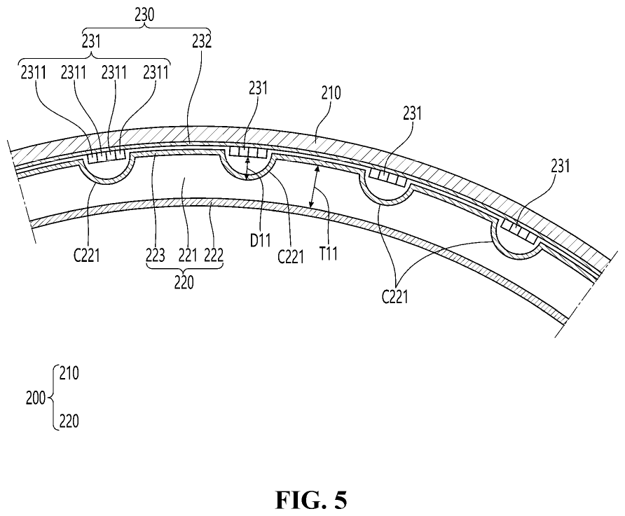

As shown in FIG. 5, the substrate 221 of the internal mask unit 220 may include a material that transmits light so that the light of the light source unit 230 is transmitted so as to be irradiated toward the user's face. For example, the substrate 221 may be formed of transparent plastic.

The outer surface (i.e., the surface adjacent to the user's face) of the substrate 221 is a flat surface, while the inner surface (i.e., the surface opposite to the outer surface and adjacent to the external mask unit 221) may be a concavo-convex surface having a plurality of concave portions C221.

In this case, the inner surface of the substrate 221 between the two adjacent concave portions C221 may have a flat surface.

In this case, the inner surface of the substrate 221 of the internal mask unit 220 may have concave portions C221 located at a predetermined interval in a horizontal direction of the substrate 221, which is a width direction of the face.

In addition, the concave portions C221 may be located at predetermined intervals even in a longitudinal direction of the substrate 221, which is a longitudinal direction of the face.

As a result, the substrate 221 has a shape in which a plurality of planoconcave lenses are located adjacent to each other, and thus, the substrate 221 may serve as a planoconcave lens.

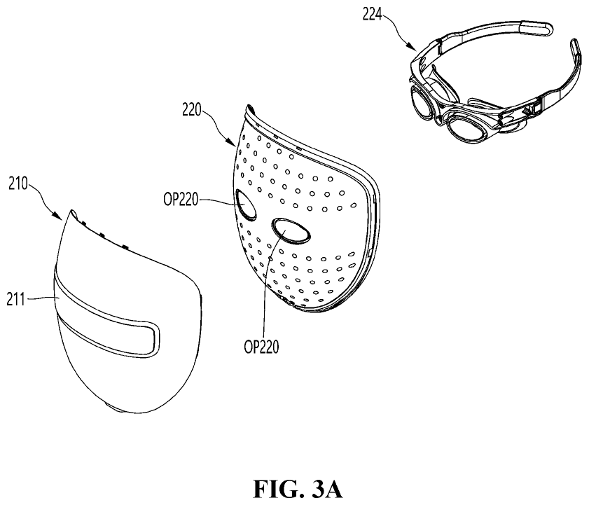

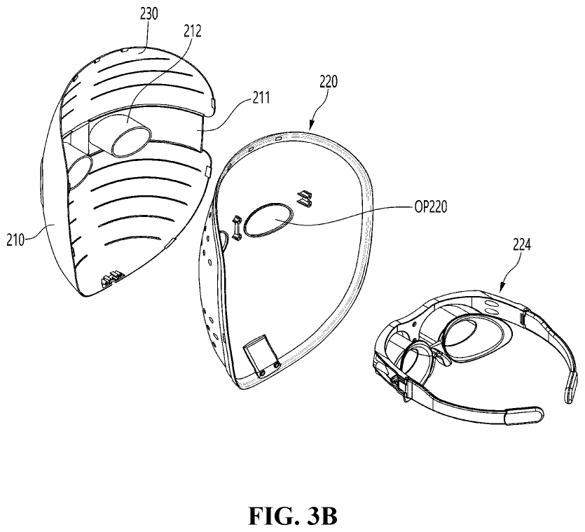

In this case, a periphery of the pair of openings OP220 may be a flat surface on which the concave portion C221 is not located. Accordingly, as shown in FIG. 3A, a band portion extending in a band shape in a horizontal direction from the pair of openings OP220 may be a flat surface without the concave portion C221. A pair of openings OP220 may be located in the band portion.

Accordingly, the concave portion C221 may be arranged in a predetermined shape on the inner surface of the substrate 221.

Each of these concave portions C221 may be located to correspond to each light source 231.

Here, the corresponding light source 231 may be located within each concave portion C221, and the light source 231 may irradiate light toward the corresponding concave portion C221.

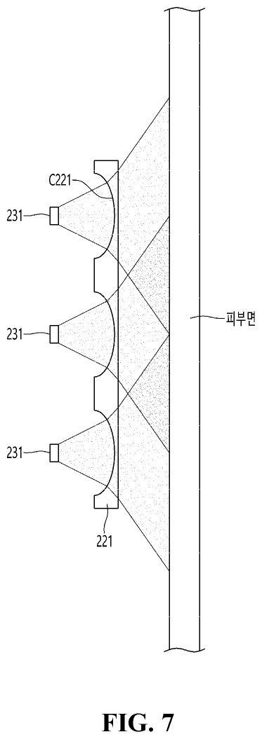

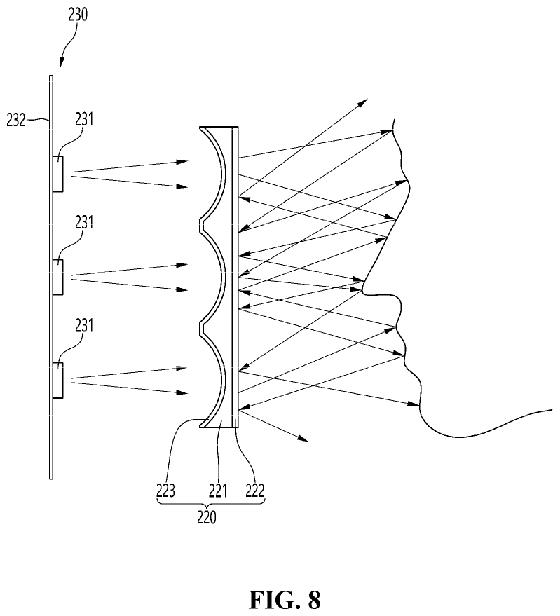

Therefore, when light is irradiated from the corresponding light source 231, each concave portion C221 diffuses incident light so as to be irradiated toward the user's face as shown in FIG. 7, and thus, the concave portion C221 may function as a diffusion lens diffusing incident light.

For the light diffusion function, a surface of each concave portion C221 facing each light source 231 may have a concave curved surface as shown in FIG. 5. Each light source 231 may be spaced apart from the surface of the concave portion C221 by a predetermined distance without contacting the surface of the concave portion C221 so that each of the concave portions C221 may function as a diffusion lens.

As such, each light source 231 faces the concave surface of the corresponding concave portion C221, so that light output from each light source 231 may be entirely irradiated toward the corresponding concave portion C221 which faces.

Therefore, as shown in FIG. 7, light output from each light source 231 is incident toward the corresponding concave portion C221 performing a diffusion lens function, and a beam of light irradiated by the surface of the concave portion C221 is diffused and spread and output toward the user's face through the internal mask unit 220.

Accordingly, light diffused by the concave portion C221 is output while uniformly spreading over the entire user's face, so that uniformity of a light intensity irradiated to the user's face is improved.

In general, when the portion of the substrate 221 facing each light source 231 does not have a diffusion lens function, for example, in the case of a flat surface, light output from each light source unit 231 may be irradiated to the user's face directly through a corresponding portion of the substrate 221.

In this case, when the light output from each light source 231 is irradiated to the surface, the intensity of the light may decrease from the center portion to the edge portion of the irradiation surface.

In addition, even stronger light may be irradiated to the portion of the irradiation surface directly facing each light source 231 than an otherwise portion (for example, a portion of the irradiation surface located between two adjacent light sources 231).

As the irradiation intensity of light according to the positions is different, the intensity of light and the amount of light irradiated to the skin facing the central portion of each light source 231 may be much stronger and greater than the skin of the otherwise portion.

Therefore, the intensity and amount of light irradiated to the face are not uniform, and there may be a risk of skin damage or burns in the face part irradiated with a stronger and larger amount of light.

Conversely, the amount of light irradiated for skin improvement is weak and insufficient in the face part irradiated with a relatively small intensity and a small amount of light, so that the skin improvement effect is not substantially exhibited or the improvement effect may be reduced.

However, as in this example, since the light output from each light source 231 is diffused and spread by the light diffusion function of the concave portion C221, light passing through the internal mask unit 220 is uniformly distributed in intensity and amount and then made incident to the user's face, thereby generating an effect in which the amount and intensity of light incident on the entire face of the user becomes uniform.

Here, a radius of curvature of each concave portion C221 may be 1 mm to 2 mm, and by the radius of curvature of the concave portion C221, each concave portion C221 performs a more efficient light diffusion function to improve uniformity of the light irradiated to the user's face surface.

A distance D11 between the concave portion C221 of the substrate 221 and the irradiation surface of the light sources 231 corresponding to each other may be 0.5 mm to 2 mm.

Accordingly, as the distance D11 between the light source 231 and the concave portion C221 is maintained between 0.5 mm and 2 mm, the concave portion C221 may efficiently perform the light diffusion function of light output from the corresponding light source 231.

In addition, a thickness of the substrate 221 is preferably reduces a weight of the substrate 221, without adversely affecting the transmission of light, to minimize user's inconvenience.

In this example, a maximum thickness T11 of the substrate 221 may be 2 mm to 3 mm. As such, when the maximum thickness T11 of the substrate 221 is 2 mm to 3 mm, the transmittance of light transmitted through the substrate 221 and irradiated toward the user's skin is maintained to the maximum, so that the thickness T11 of the substrate 221 may be minimized. In this case, transmittance of light with respect to the substrate 221 may be 80 to 90%.

The reflective layer 222 located on the outer surface of the substrate 221 may be formed by applying a reflective material that reflects light.

Since the reflective layer 222 is supposed to allow light output from the light source unit 230 to be transmitted therethrough, the reflective layer 222 may be formed of a reflective material (e.g., a transparent reflective material) through which light is transmitted.

In general, when light is output from the light source unit 230 and incident on the user's face, only a portion (about 40% to 50%) of the light that passes through the substrate 221 and is incident toward the user's face is introduced into the skin and the rest is reflected from the face and returned to the internal mask unit 220.

Therefore, as in this example, as the reflective layer 222 is located on the outer surface of the substrate 221 of the internal mask unit 220 facing the face, the operation in which the light returning toward the internal mask unit 220 after being reflected from the face is reflected by the reflective layer 222 and re-incident on the face may be repeated a plurality of times.

Thus, compared with a case in which the reflective layer 222 is not present, when the reflective layer 222 is present on the substrate 221, the amount of light incident on the user's face is significantly increased, and accordingly, the amount of light introduced into the skin is also increased, thereby significantly increasing the skin improvement effect.

In addition, due to the diffusion function of the light by the substrate 221, even if at least one of the intensity and amount of light incident on the user's skin decreases, the amount of light finally introduced into the skin is increased by the reflective layer 222, and thus, the skin improvement effect may not be decreased but rather increased.

In this case, the thickness of the reflective layer 222 may be any thickness capable of exhibiting a reflective effect without adversely affecting the transmission of light, and may be, for example, 10 μm to 20 μm.

In addition, a distance from the surface of the reflective layer 222 to the user's skin may be approximately 1.5 cm to 2.5 cm, and in this case, user wearability may be improved, while increasing the amount of light reflected toward the user's skin by the reflective layer 222.

The photocatalyst layer 223 located on the inner surface of the substrate 221 may contain at least one type of transition metal fine particles.

In this case, the transition metal fine particles may contain not only a transition metal but also a transition metal compound. Accordingly, the transition metal fine particles may contain at least one of a transition metal and a transition metal compound.

Examples of such transition metal fine particles may include titanium (Ti), silver (Ag), platinum (Pt), titanium dioxide, tungsten (WO₃), perovskite-type composite metal oxide, cadmium selenide (CdSe), zinc oxide (ZnO), cadmium sulfide (CdS), strontium titanate (SrTiO₃; Perovskite type), potassium iodate (KNbO₃), gallium arsenide, cadmium sulfide or iron oxide, and zirconia (ZrO₂).

In this example, a volume average particle diameter of the transition metal fine particles may be 1 μm or less, for example, 0.0001 μm to 1 μm, or 0.01 μm to 0.5 μm, or 0.05 μm to 0.3 μm.

Since the light from the light source unit 230 should be irradiated toward the user's face, the photocatalyst layer 223 may also be formed of a material through which light is transmitted.

Such a photocatalyst layer 223 may exhibit a skin improvement effect, a skin disinfection effect, etc. depending on the type of transition metal contained therein.

For example, in a case in which the photocatalyst layer 223 contains titanium (Ti), when the photocatalyst layer 223 is irradiated with light and heat from the facing light source unit 230, the transition metal particles are excited and activated by the irradiated light and heat to generate a magnetic field, and the generated magnetic field is applied to the user's face.

Therefore, by the magnetic field applied toward the skin of the face, a skin improvement effect based on blood circulation of the skin, etc. may be obtained.

Accordingly, at least one of a user's skin improvement effect, a skin disinfection effect, and a skin purification effect may be exhibited by the function of the photocatalyst layer 223.

In the case of FIG. 5, the photocatalyst layer 223 is located on the inner surface of the substrate 221, but alternatively, the photocatalyst layer 223 may be located between the outer surface of the substrate 221 and the reflective layer 222.

In addition, the photocatalyst layer 223 may be located on one of the inner surface or the outer surface of the substrate 221 or may be located on both the inner surface and the outer surface of the substrate 221.

A thickness of the photocatalyst layer 223 may also be 10 μm to 20 μm, and in this case, the photocatalyst layer 223 may efficiently perform the corresponding function without adversely affecting the transmission of light.

As already described above, as shown in FIG. 3, the internal mask unit 220 includes a pair of openings OP220 exposing the user's eyes, and by exposing the user's eyes through the opening OP 220, the user's eyes may be protected from light irradiated from the light source unit 230

The opening OP222 may be blocked with the protective plate 211 such as transparent plastic or the like as shown in FIG. 2.

The wearing unit 224 mounted on the face mask unit 220 is configured as glasses and includes a main body 2241 mounted on the edge of the opening OP220 located in the internal mask unit 220 and wearing legs 2242 located on both sides of the main body 2241.

The main body 2241 may be mounted by being inserted into a pair of protrusions 212 of the external mask unit 210. Accordingly, the pair of protrusions 212 of the external mask unit 210 may be a wearing unit mounting protrusion for mounting the wearing unit 224.

Here, an interval corresponding to at least a thickness of the main body 2241 is maintained between the outer surface of the internal mask unit 220 and the user's skin, so that the user's skin is separated from the outer surface (i.e., the surface of the reflective layer 222) of the internal mask unit 220 by at least the thickness of the main body 2241.

In this case, the distance between the internal mask unit 220 and the user's skin, that is, the thickness of the main body 2241 may be determined according to the intensity and focal length of light output from the light source 231.

Therefore, since the distance between the light source 231 and the skin is adjusted according to the thickness of the main body 2241, at least one of an arrival distance for light output from the light source 231 to reach the skin, a depth of light penetration into the skin, and an irradiation area of light may be adjusted using the thickness of the main body 2241.

In addition, as the wearing unit 224 in the form of glasses is located in the internal mask unit 220, the user may wear the face mask 200 by mounting the wearing legs 2242 on the ears as if wearing glasses, thereby improving convenience of wearing.

In addition, in a state in which the wearing legs 2242 are normally mounted on the ear, there is no problem in the wearing state, such as the worn face mask 200 falling off, or the like, unless a large shock is applied from the outside.

Therefore, the user may walk normally even in a state of wearing the face mask 200, rather than lying in bed or sitting on a chair for a predetermined time, and thus, discomfort after wearing is also significantly reduced.

As shown in FIG. 7, the light source unit 230 located between the external mask unit 210 and the internal mask unit 220 may include a flexible substrate (e.g., a flexible printed circuit board (FPCB)) 232 on which a plurality of light sources 231 are mounted.

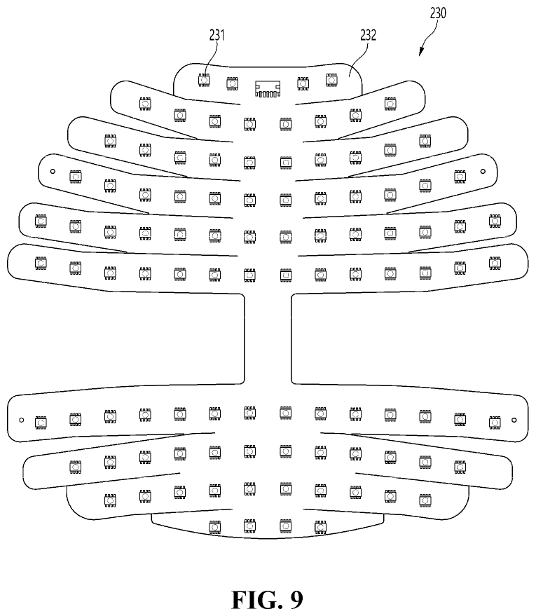

Accordingly, the light source unit 230 may be located to be attached to the inner surface of the external mask unit 210. An example of the planar shape of the light source unit 230 is shown in FIG. 9.

As shown in FIG. 9, the planar shape of the flexible substrate 232 constituting the light source unit 230 may be determined based on the shape of a user's face, and the light source unit 230 may not be located around the user's eyes and the risk of damage to the eyes due to the light irradiated from the light source unit 230 may be prevented.

In FIG. 9, the light source 231 is manufactured in the form of a semiconductor chip and mounted on the flexible substrate 232.

As already described above, the plurality of light sources 231 may be located to correspond to the concave portion C221 of the internal mask unit 220, so that light output from each light source 231 may be efficiently irradiated into the concave portion C221.

Each of these light sources 231 may include at least one light emitting device 2311 for outputting at least one type of light.

In this example, the light emitting device 2311 may be a light emitting diode (LED), and the light emitting device 231 provided in one light source unit 230 may be at least one of a red light emitting device (e.g., red LED) that outputs red light, a yellow light emitting device (e.g., yellow LED) that outputs yellow light, a green light emitting device (e.g., green LED) that outputs green light, and an infrared light emitting device (infrared (IR) LED) that outputs infrared light.

In this case, operating frequencies of driving signals for driving the red light emitting device, the yellow light emitting device, the green light emitting device, and the infrared light emitting device may all be the same and may be, for example, 130 Hz to 170 Hz.

However, at least one light emitting device (e.g., the red light emitting device) may have an operating frequency different from those of other light emitting devices, and in this case, the at least one light emitting device (e.g., the red light emitting device) may have an operating frequency of 50 Hz to 90 Hz, and the operating frequencies of the other light emitting devices (e.g., the yellow light emitting device, the green light emitting device, and the infrared light emitting device) may be 130 Hz to 170 Hz.

Red light output from the red light emitting device 2311 may stimulate activity of fibroblasts in the skin, thereby exerting an effect on the regeneration of collagen and elastine and wound recovery.

Yellow light output from the yellow light emitting device 2311 exerts an excellent effect on skin regeneration, prevents occurrence of wrinkles to help restore facial skin and also exhibits wound recovery and whitening effects.

In addition, green light output from the green light emitting device 2311 may help repair and maintain the skin by inducing cell growth, thereby alleviating dark spots on the skin surface and helping to brighten the skin.

Infrared light output from the infrared light emitting device 2311 may be effective in alleviating pain, relaxing muscles, promoting blood circulation, and removing wastes.

As already described above, the face mask 200 of this example includes the reflective layer 222 for a repeated reflective operation toward the user's face, so that the amount and intensity of light incident toward the user's face may be increased.

Even if the number of light sources 231 used in the light source unit 230 is reduced by the function of the reflective layer 222, the amount and intensity of light incident toward the user's face are not reduced.

Therefore, since the face mask 200 according to the present example reduces the number of light sources 231 in use, manufacturing cost may be reduced and space for an installation of the light sources 231 may be increased due to the reduced light sources 231.

In this example, an interval between the light sources 231 adjacent in an up-down direction (i.e., in a vertical direction) may be approximately 20 mm to 25 mm, and an interval between two light sources 231 adjacent in a left-right direction (or in a horizontal direction) may be 18 mm to 22 mm.

Also, as an example, the total number of light sources 231 provided in the light source unit 230 may be 80 to 100.

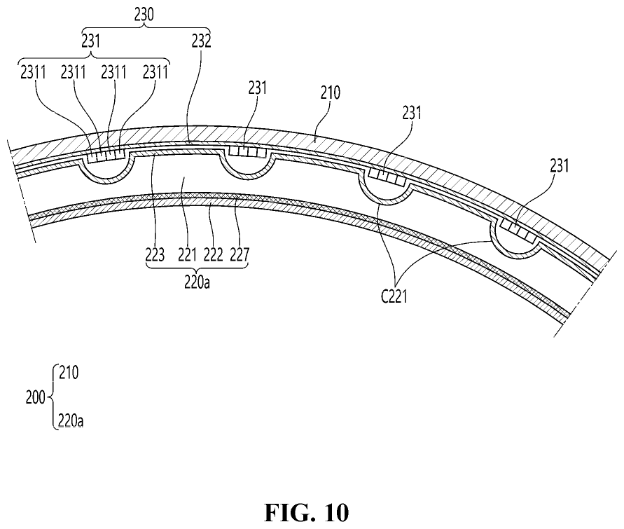

FIG. 10 shows an internal mask unit 220a according to another example.

As shown in FIG. 10, the internal mask unit 220a according to another example may have the same structure as the internal mask unit 220 illustrated in FIG. 5, except for a UV blocking layer 227 located between the substrate 221 and the reflective layer 222, when compared with the internal mask unit 220 illustrated in FIG. 5.

Therefore, since the ultraviolet component of light irradiated into the user's skin is blocked by the UV blocking layer 227, skin damage due to ultraviolet rays may be additionally prevented.

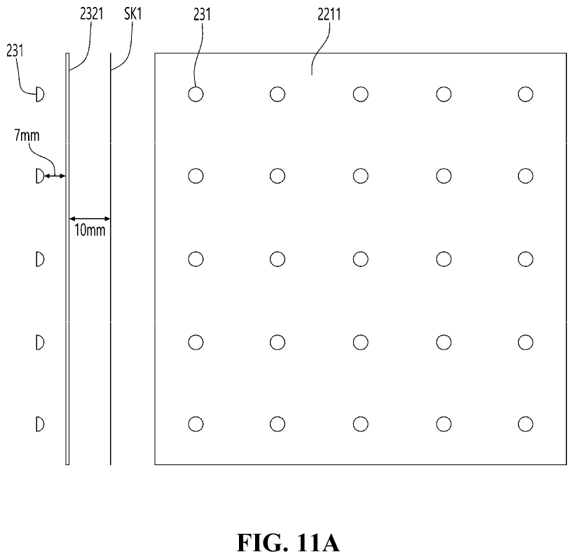

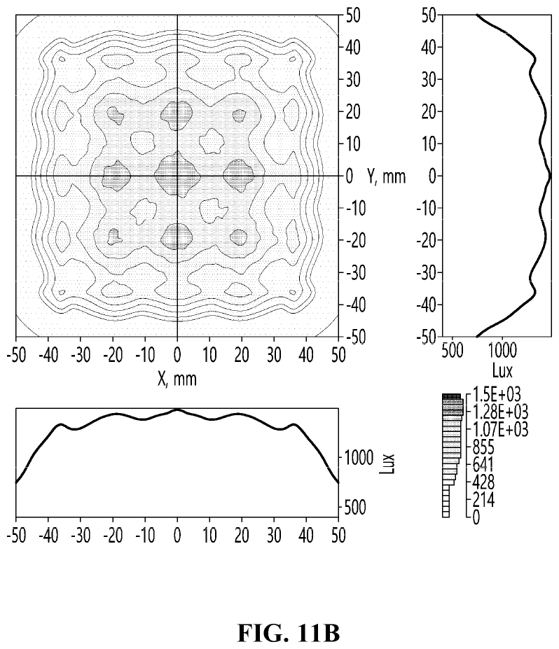

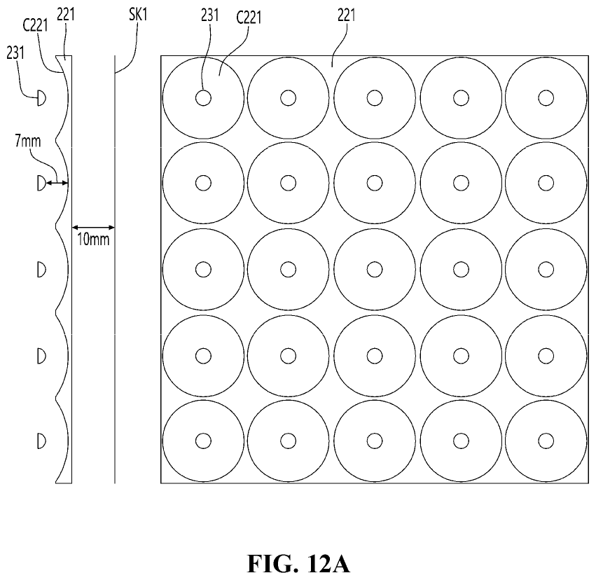

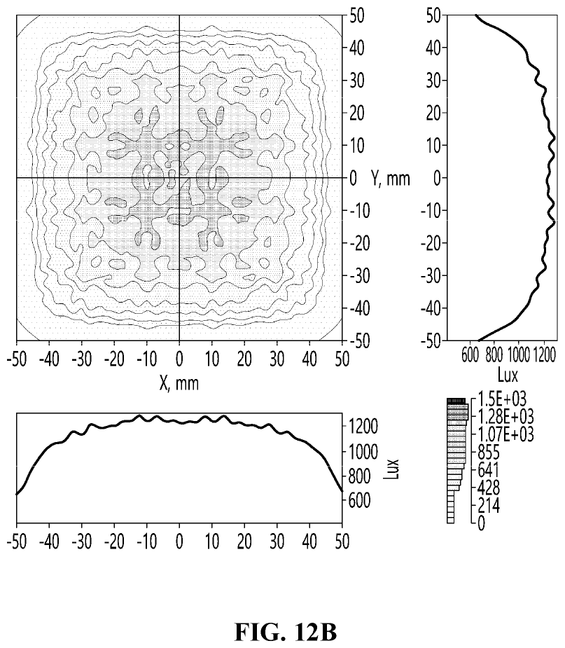

Next, changes in light uniformity in Comparative Examples in which a surface of a substrate on which light is incident is flat without a concave portion and Examples of the present disclosure including an uneven surface having a concave portion will be described with reference to FIGS. 11A to 12B.

As shown in FIGS. 11A and 12A, in Comparative Examples and Examples, the shapes and sizes (100 mm×100 mm) of the substrates 2211 and 221 were the same, the number of light emitting devices (LEDs) 231 as light sources arranged in the substrates 2211 and 221 were a total of 25 (width×length=5×5), and power output from the total light emitting devices was 17.5 lm. A Z-axis position of a lens for imaging the substrates 2211 and 221 was 7 mm from the substrates 2211 and 221, and a thickness of the lens was 1 mm. In addition, a length of the overall optical system was 18 mm.

In addition, a distance from each light emitting device 231 to the substrates 2211 and 221 was 7 mm, and a distance from the substrates 2211 and 221 to a corresponding skin surface SK1 was 10 mm.

In addition, as shown in FIGS. 11A and 12A, distances of the two optical devices 231 adjacent to each other in the horizontal and vertical directions were the same.

FIGS. 11A and 11B show a case of Comparative Examples in which the corresponding surface of the substrate 2211 on which light is incident has a flat surface without a concave portion, and FIGS. 12A and 12B show Examples of the present disclosure in which the concave portion C221 is provided on the surface of the substrate 221.

Under these conditions, images of the substrates 2211 and 221 captured by an operation of an optical system when 25 light emitting devices installed on the substrates 2211 and 221 were turned on correspond to FIGS. 11B and 12B.

As shown in FIGS. 11B and 12B, it can be seen that uniformity of light is improved in the case in which a plurality of concave portions C221 are located on the substrate 221 (FIG. 12B), compared with the otherwise case (FIG. 11B), as can be confirmed from the captured images.

In the case of Comparative Examples and Examples, uniformity of light may be calculated numerically as shown in [Table 1] and [Table 2]. In the table below, an average is an average value of the obtained illuminance Lux, a minimum value MIN is a minimum value among the obtained illuminances, and a maximum value MAX is a maximum value among the obtained illuminances.

TABLE 1
100 × 100 mm Simulation Result

| Max. Spatial Luminance | 1496.4 Lux |
|---|---|
| Luminous efficiency | 66.4% |
| Uniformity formula: | 34% |
| $\frac{MIN}{Average} \times 100 \%$ | |
| Uniformity formula: | 26% |
| $\frac{MIN}{MAX} \times 100 \%$ | |

TABLE 2
100 × 100 mm Simulation Result

| Max. Spatial Luminance | 1305 Lux |
|---|---|
| Luminous efficiency | 58.5% |
| Uniformity formula: | 40% |
| $\frac{MIN}{Average} \times 100 \%$ | |
| Uniformity formula: | 31% |
| $\frac{MIN}{MAX} \times 100 \%$ | |

As shown in [Table 1] and [Table 2], it can be seen that the uniformity of light of Comparative Examples was 34% and 26%, whereas uniformity of light of Examples increased to 40% and 31%, respectively.

In addition, in the case of second Comparative Example and second Example in which the size of the substrate was changed to 60 mm×60 mm under the conditions of Comparative Examples and Examples described above, the calculated uniformity of light is as shown in [Table 3] and [Table 4].

TABLE 3
60 × 60 mm Simulation Result

| Max. Spatial Luminance | 1496.4 Lux |
|---|---|
| Luminous efficiency | 28% |
| Uniformity formula: | 68.3% |
| $\frac{MIN}{Average} \times 100 \%$ | |
| Uniformity formula: | 78.6% |
| $\frac{MIN}{MAX} \times 100 \%$ | |

TABLE 4
60 × 60 mm Simulation Result

| Max. Spatial Luminance | 1305 Lux |
|---|---|
| Luminous efficiency | 24.7% |
| Uniformity formula: | 90% |
| $\frac{MIN}{Average} \times 100 \%$ | |
| Uniformity formula: | 83.5% |
| $\frac{MIN}{MAX} \times 100 \%$ | |

Also, in this case, as shown in [Table 3] and [Table 4], the uniformity of light of second Comparative Examples was 68.3% and 78.6%, whereas the uniformity of light of second Example was increased to 90% and 83.5%, respectively.

As such, as in this example, since the plurality of concave portions C221 for light scattering are located on the substrate 221 of the internal mask unit 220, the uniformity of light irradiated toward the user's skin is improved, whereby light is uniformly irradiated to the entire face. As a result, it can be seen that stable skincare is achieved and the skin improvement effect is also improved.

Returning back to FIG. 1, the operating unit 300 serves to control the operation of the mask device 1 for skincare using light.

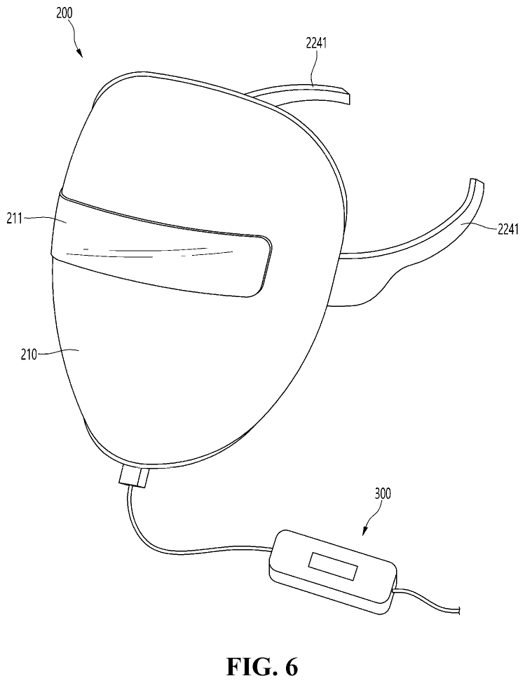

As shown in FIG. 6, the operating unit 300 may include a driving button electrically connected to the face mask 200 and operating the face mask 200, a selection button for inputting a user select option such as a skin type, an irradiation purpose, and the like, at least one indicator light indicating a state, a control circuit, and etc.

Accordingly, the user may control at least one of whether the light source unit 230 mounted on the face mask 200 is turned on, a light emission time, and a state of the output light using the operating unit 300.

The mask device 1 for skincare using light of this example may control an operation of the light source unit 230 according to a result of user selection such as a skin type (e.g., oily skin, dry skin, or combination skin) and a light irradiation purpose (e.g., for wound recovery, skin elasticity, skin regeneration or whitening).

Therefore, when the user inputs his or her skin type and irradiation purpose using the selection button of the operating unit 300, the user's selection result is applied to the control circuit.

Therefore, the control circuit may determine the skin type and the irradiation purpose selected by the user using the applied signal and control a light emission type (e.g., different types of light mixing ratio) and light emission time of the light source unit 230 according to the determination result.

So far, embodiments of the mask device for skincare using light of the present disclosure have been described. The present disclosure is not limited to the embodiments described above and the accompanying drawings, and various modifications and variations may be made from the point of view of those of skilled in the art to which the present disclosure pertains. Accordingly, the scope of the present disclosure should be defined not only by the claims of the present disclosure, but also by those claims and their equivalents.

1. A mask device for skincare using light comprising:
an external mask unit;
an internal mask unit located to be spaced apart from the external mask unit;
a plurality of light sources located between the external mask unit and the internal mask unit and configured to output light toward the internal mask unit,
wherein the internal mask unit includes a substrate including a first surface having a plurality of concave portions and a second surface located opposite to the first surface, and the plurality of light sources are located to face the plurality of concave portions, respectively.

2. The mask device of claim 1, wherein the plurality of concave portions have a light diffusing function.

3. The mask device of claim 2, wherein the substrate is formed of a material that transmits light.

4. The mask device of claim 1, wherein the internal mask unit further comprises a photocatalyst layer located on at least one of the first surface and the second surface of the substrate.

5. The mask device of claim 4, wherein the photocatalyst layer contains at least one transition metal fine particle.

6. The mask device of claim 5, wherein the at least one transition metal fine particle contains at least one of a transition metal and a transition metal compound.

7. The mask device of claim 4, wherein the internal mask unit further comprises a reflective layer located on the second surface of the substrate.

8. The mask device of claim 7, wherein the internal mask unit further comprises a UV blocking layer located between the second surface of the substrate and the reflective layer.

9. The mask device of claim 1, wherein the internal mask unit further comprises a reflective layer located on the second surface of the substrate.

10. The mask device of claim 9, wherein the mask device further comprises a UV blocking layer located between the second surface of the substrate and the reflective layer.

11. The mask device of claim 1, wherein the second surface of the substrate is a flat surface.

12. The mask device of claim 1, wherein each of the plurality of light sources may include at least one light emitting device.

13. The mask device of claim 12, wherein the light emitting device is one of a red light emitting device, a green light emitting device, a yellow light emitting device, and an infrared light emitting device.

14. The mask device of claim 13, wherein operating frequencies of the red light emitting device, the green light emitting device, the yellow light emitting device, and the infrared light emitting device are all be the same.

15. The mask device of claim 14, wherein the operating frequency is 130 Hz to 170 Hz.

16. The mask device of claim 13, wherein at least one of the red light emitting device, the green light emitting device, the yellow light emitting device, and the infrared light emitting device has an operating frequency different from those of the other light emitting devices.

17. The mask device of claim 15, wherein the operating frequency of the red light emitting device is 50 Hz to 90 Hz, and the operating frequencies of the green light emitting device, the yellow light emitting device, and the infrared light emitting device are 130 Hz to 170 Hz.

18. The mask device of claim 1, further comprising:
a UV blocking layer located on the second surface of the substrate.
